(19) United States
(12) Patent Application Publication
(10) Pub. No.: US 2009/0171110 A1
(43) Pub. Date: Jul. 2, 2009

(54) PROCESS FOR PREPARING N-METHYL-3, 4-DIMETHOXYPHENYLETHYLAMINE

(76) Inventors: Davide ALBANI, Merate (LC) (IT); Roberto Arosio, Civate (LC) (IT); Valeriano Merli, Cremella Lecco (IT)

Correspondence Address: KENYON & KENYON LLP, ONE BROADWAY, NEW YORK, NY, 10004, US

(21) Appl. No.: 12/344,012

(22) Filed: Dec. 24, 2008

Related U.S. Application Data
(60) Provisional application No. 61/016,704, filed on Dec. 26, 2007.
(60) Provisional application No. 61/058,403, filed on Jun. 3, 2008.
(60) Provisional application No. 61/078,036, filed on Jul. 3, 2008.
(60) Provisional application No. 61/137,243, filed on Jul. 28, 2008.
(60) Provisional application No. 61/188,686, filed on Aug. 11, 2008.
(60) Provisional application No. 61/103,064, filed on Oct. 6, 2008.

(51) Int. Cl. C07C 253/00; C07C 209/00

(52) U.S. Cl. 558/308; 564/374

(57) ABSTRACT

Provided are intermediates useful for the preparation of verapamil and methods for their preparation.

### CROSS REFERENCE TO RELATED APPLICATIONS

The present invention claims the benefit of the following U.S. Provisional Patent Application Nos.: 61/016,704, filed Dec. 26, 2007; 61/058,403, filed Jun. 3, 2008; 61/078,036, filed Jul. 3, 2008; 61/137,243, filed Jul. 28, 2008; 61/188,686, filed Aug. 11, 2008; and 61/103,064, filed Oct. 6, 2008. The contents of these applications are incorporated herein by reference.

### FIELD OF INVENTION

The present invention relates to a process for preparing N-methyl-3,4-dimethoxyphenylethylamine and a salt thereof, an intermediate of Verapamil.

### BACKGROUND OF THE INVENTION

Verapamil, 2-(3,4-dimethoxyphenyl)-5-[2-(3,4-dimethoxyphenyl)ethyl-methyl-amino]-2-(1-methylethyl) pentanenitrile of the following formula

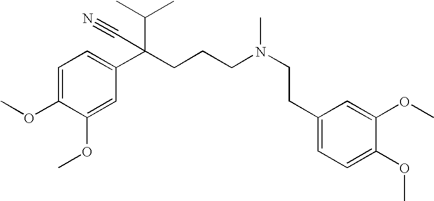

is a drug that has been used in the treatment of hypertension, angina pectoris, cardiac arrhythmia, and most recently, for headaches. Verapamil has also been used as a vasodilator during cryopreservation of blood vessels. It is a class 4 antiarrhythmic, more effective than digoxin in controlling ventricular rate, and was approved by the FDA in 1981.

Verapamil is marketed under the trade name Calan® by Knoll A. G. The process for preparation of verapamil is disclosed in BE615861. This process is done by alkylation of veratryl cyanide with (N-Methyl-N-Homoveratryl)-γ-amino-chloropropane by means of sodium amide in toluene at reflux temperature. Then, the obtained product is further alkylated with isopropyl bromide by means of sodium amide in toluene, obtaining Verapamil. Alternatively the two alkylation steps can be reverted.

Verapamil and its preparation are also described in U.S. Pat. No. 3,261,859.

One of the methods to prepare Verapamil is via a hydrochloride salt of N-Methyl 3,4-dimethoxyphenylethylamine of the following formula,

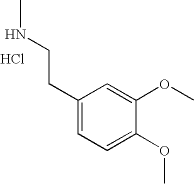

as reported in U.S. Pat. No. 4,115,432 and in U.S. Pat. No. 4,350,636.

According to the above patents, the hydrochloride salt of N-Methyl 3,4-dimethoxyphenylethylamine can be prepared by reduction of the corresponding ketone or alcohol analogues according to the following scheme:

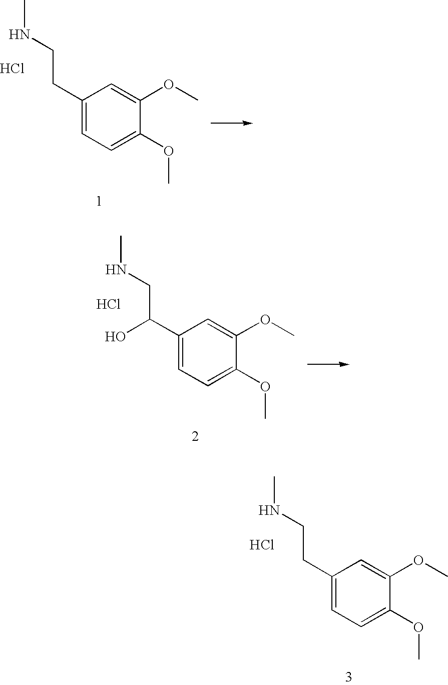

The reduction of the ketone of formula 1 to the alcohol of formula 2 is described in several publications.

U.S. Pat. No. 2,683,743 reports the reduction of the ketone to the alcohol using Raney Nickel catalyst. GB patent No. 684,781 reports the reduction of the ketone to the alcohol using PdCl₂ catalyst. The Journal of the American Chemical Society 1955, 77, 5757 reports the same reduction but of a benzyl protected amine derivative of the ketone using Pd/C catalyst.

The Journal of the American Chemical Society 1949, 71, 3419 reports an unsuccessful attempt to prepare the compound of formula 3 by reducing the above benzyl protected amine derivative of the ketone using PtO₂ catalyst.

Thus, there is a need in the art for a method to prepare the salt of N-Methyl 3,4-dimethoxyphenylethylamine from the ketone of formula 1 or from the alcohol of formula 2.

### SUMMARY OF THE INVENTION

In one embodiment, the invention encompasses a process for preparing N-Methyl-3,4-dimethoxyphenylethylamine and salt thereof of formula 6.

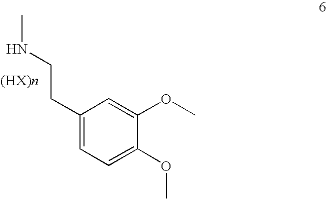

comprising reacting N-methyl-2-(3,4-dimethoxyphenyl)-2-oxy-ethylamine and salt thereof of formula 4

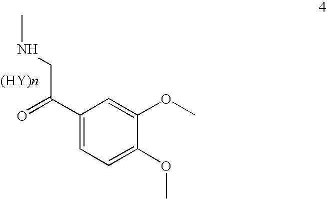

or N-methyl-2-(3,4-dimethoxyphenyl)-2-hydroxy-ethylamine and salt thereof of formula 5

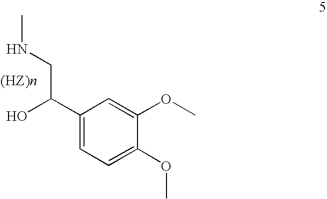

with hydrogen gas, a palladium hydrogenation catalyst and a Lewis acid; wherein n is either 0 or 1, when n of HY or HZ is 0 the reaction comprises also HCl or HBr; and HX, HY and HZ are independently an acid selected from a group consisting of: HCl and HBr, and combination thereof.

In one embodiment, the invention encompasses a process for preparing verapamil of the following formula

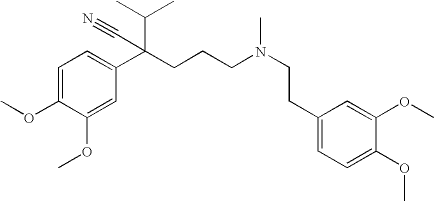

by a process comprising preparing a salt of N-Methyl-3,4-dimethoxy phenylethylamine of formula 6 by either one of the above processes and converting it to Verapamil.

### BRIEF DESCRIPTION OF THE DRAWINGS

FIG. 1 illustrates a powder X-ray diffraction pattern of crystalline form of N-Methyl-3,4-dimethoxyphenylethylamine hydrochloride.

### DETAILED DESCRIPTION OF THE INVENTION

The present invention relates to an improved process for preparing Verapamil intermediate, N-Methyl-3,4-dimethoxyphenylethylamine and salt thereof of formula 6 from N-methyl-2-(3,4-dimethoxyphenyl)-2-oxy-ethylamine and salt thereof of formula 4, without the need to isolate the intermediate N-methyl-2-(3,4-dimethoxyphenyl)-2-hydroxy-ethylamine and salt thereof of formula 5. The direct conversion of N-methyl-2-(3,4-dimethoxyphenyl)-2-oxy-ethylamine and salt thereof of formula 4 to N-Methyl-3,4-dimethoxyphenylethylamine and salts thereof of formula 6 is possible due to the presence of a Lewis acid, see for example, Examples 5 and 7 versus Examples 6 and the rest. Accordingly, the current process allows the isolation of N-methyl-2-(3,4-dimethoxyphenyl)-2-hydroxy-ethylamine and salt thereof of formula 5 to be skipped and thus produces less waste. Also, the obtained compound of formula 6 may be purified via a simple base-salt transformation providing a purified compound of formula 6 having a purity of at least 99% area by HPLC. Accordingly, the process is suitable for large scale production.

The process can be illustrated by the following scheme:

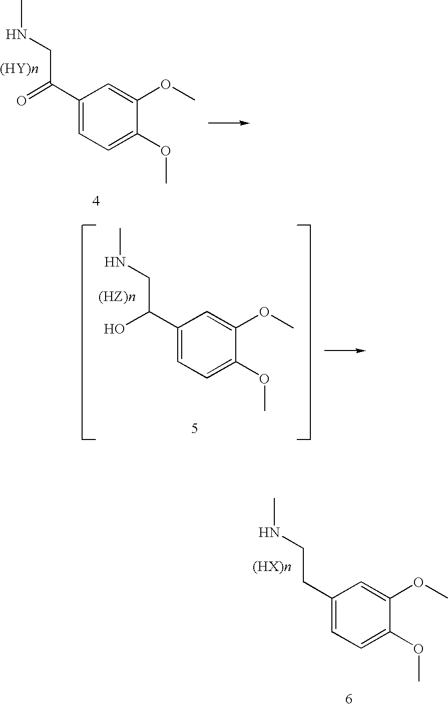

wherein n is either 0 or 1, and HX, HY and HZ are independently an acid selected from a group consisting of: HCl and HBr, and combination thereof.

N-Methyl-3,4-dimethoxyphenylethylamine and a salt thereof of formula 6

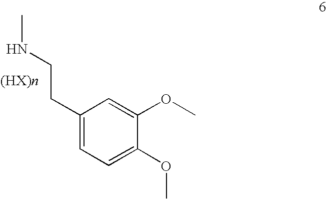

can be prepared by a process comprising reacting N-methyl-2-(3,4-dimethoxyphenyl)-2-oxy-ethylamine and a salt thereof of formula 4

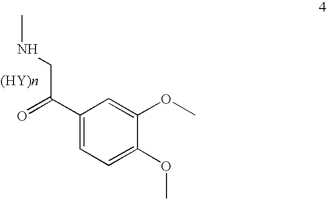

with hydrogen gas, a palladium hydrogenation catalyst and a Lewis acid; wherein n is either 0 or 1, when n of HY is 0 the reaction comprises also HCl or HBr; and HX and HY are independently an acid selected from the group consisting of: HCl and HBr, and a combination thereof.

The starting hydrochloride salt of N-methyl-2-(3,4-dimethoxyphenyl)-2-oxy-ethylamine and other salts of formula 4 can be prepared by the Houben-Hoesch reaction for example as disclosed in Example 2. This procedure can be used also to prepare the hydrobromide salt of N-methyl-2-(3,4-dimethoxyphenyl)-2-oxy-ethylamine by using HBr instead of HCl. If desired, the salt can be converted to the free base form, i.e., N-methyl-2-(3,4-dimethoxyphenyl)-2-oxy-ethylamine, by reacting the salt with a base.

The starting N-methyl-2-(3,4-dimethoxyphenyl)-2-oxy-ethylamine and salt thereof of formula 4 can be isolated at the end of the Houben-Hoesch reaction or can be used without isolation, as exemplified in Example 2. When used without isolation, it is present in an aqueous mixture that is obtained from the work-up process of the Houben-Hoesch reaction. Preferably, the starting N-methyl-2-(3,4-dimethoxyphenyl)-2-oxy-ethylamine and a salt thereof of formula 4 are used without isolation.

When n is 0, i.e., the free base, i.e., N-methyl-2-(3,4-dimethoxyphenyl)-2-oxy-ethylamine is used as a starting material, the reaction comprises also HCl or HBr. Preferably, n is 1, i.e., the compound of formula 4 is the salt of N-methyl-2-(3,4-dimethoxyphenyl)-2-oxy-ethylamine. Preferably, the salt is a hydrochloride salt.

Preferably, the aqueous mixture of the starting N-methyl-2-(3,4-dimethoxyphenyl)-2-oxy-ethylamine and salt thereof of formula 4 contains also a Lewis acid. The Lewis acid can be combined with the aqueous mixture or can be present from the Houben-Hoesch reaction.

If used after isolation, the compound of formula 4 is preferably, combined with water and then with the Lewis acid, to provide the said aqueous mixture.

Preferably, the concentration of the Lewis acid in the aqueous mixture is of about 5% to about 15% by weight (g/g of water). More preferably, the concentration of the Lewis acid in the aqueous mixture is of about 9% to about 13% by weight (g/g of water).

Preferably, the Lewis acid is a metallic Lewis acid, more preferably, a metallic Lewis acid containing a halogen counter ion, such as Cl⁻ and Br⁻. Preferably, the metallic Lewis acid containing a halogen counter ion is selected from the group consisting of: Aluminium, Titanium, Iron and Zinc Lewis acids, more preferably, aluminium. Examples of such Lewis acids are AlCl₃, AlBr₃, FeCl₃, TiCl₄, ZnCl₂ and ZnBr₂. Preferably, the Lewis acid is FeCl₃, FeBr₃, AlCl₃ or AlBr₃, more preferably, AlCl₃.

Preferably, about 1 to about 3 mole equivalent of Lewis acid per mole equivalent of the compound of formula 4 are used. More preferably, about 1.5 to about 2.5 mole equivalent of Lewis acid per mole equivalent of the compound of formula 4 are used.

Optionally, said aqueous mixture can also comprise an acidic salt having a halogen counter ion such as Cl⁻ or Br⁻, preferably, an ammonium salt, such as NH₄Cl.

Preferably, said mixtures comprising the starting compound of formula 4, the Lewis acid, and optionally, an acid and/or an acidic salt, can be heated prior or after the addition of the palladium hydrogenation catalyst. Preferably, heating is done to a temperature of about 55° C. to about 100° C. More preferably, heating is done to a temperature of about 60° C. to about 80° C., providing a suspension. More preferably, the catalyst is added to the suspension in a “drop wise addition”. As used herein the term “drop wise addition” refers to an addition rate, for example, of about 50 mg to about 200 mg per N-methyl-2-(3,4-dimethoxyphenyl)-2-oxy-ethylamine and a salt thereof of formula 4, more preferably, of about 130 mg per hour, when 45 g of N-methyl-2-(3,4-dimethoxyphenyl)-2-oxy-ethylamine and a salt thereof of formula 4 are used. The addition rate can be modified according to the amount of the starting material.

The “palladium hydrogenation catalyst” as used herein is a palladium catalyst typical for hydrogenation reactions, such as Pd (OH)₂, PdCl₂, Pd/C Pd/graphite, Palladium on activated Charcoal or palladium catalysts that is polluted with about 5% (w/w) of Ruthenium.

Preferably, the palladium hydrogenation catalyst is Pd/C or Palladium on activated Charcoal. Preferably, the total amount of the catalyst that is added is about 0.1% to about 10% by weight of the catalyst per weight of the starting compound of formula 4 (g/g). More preferably, when the catalyst is Pd/C, the total amount of the catalyst that is added is about 5% by weight of the catalyst per weight of the starting compound of formula 4 (g/g). More preferably, when the catalyst is Pd on Charcoal, the total amount of the catalyst that is added is about 5% by weight of the catalyst per weight of the starting compound of formula 4 (g/g).

Preferably, the catalyst is wet. As used herein the term “wet” in respect to the Pd hydrogenation catalyst refers to Pd catalyst containing about 50% to about 60% by weight of water.

Further, a suspension comprising all the above reactants is then reacted with hydrogen gas. Typically, hydrogen gas is bubbled into the suspension providing the hydrogenation reaction mixture. Preferably, hydrogen gas is bubbled at a pressure of about 20 psi to about 200 psi. More preferably, hydrogen gas is bubbled at a pressure of about 60 psi to about 100 psi.

Preferably, the reaction mixture is stirred while hydrogen gas is bubbled. Preferably, the reaction mixture is stirred vigorously. As used herein the term “vigorous” refers to a stirring rate of about 300 rpm to about 400 rpm. Preferably, of about 360 rpm.

Preferably, the hydrogenation is done upon heating. Optionally, the heating can be done in two stages. Preferably, in the first stage a temperature of less than about 80° C., preferably of about 50° C. to about 70° C., more preferably of about 55° C. to about 65° C. is reached. Preferably, in the second stage, a temperature of about 80° C. to about 100° C., preferably, of about 85° C. to about 100° C. is reached.

Typically, the hydrogenation reaction can lead to the formation of the compound of N-methyl-2-(3,4-dimethoxyphenyl)-2-hydroxy-ethylamine and a salt thereof of formula 5 and then to the compound of N-Methyl-3,4-dimethoxy phenylethylamine and salts thereof of formula 6, depending on the reaction temperature.

For example, the compound of N-methyl-2-(3,4-dimethoxyphenyl)-2-hydroxy-ethylamine and salt thereof of formula 5 can be obtained in the above mentioned hydrogen pressure if the reaction is heated to a temperature of less than about 80° C., preferably, of about 50° C. to about 70° C., more preferably of about 55° C. to about 65° C.

Usually, the compound of N-methyl-2-(3,4-dimethoxyphenyl)-2-hydroxy-ethylamine and a salt thereof of formula 5 is obtained during heating for a period of about 2 hours to about 12 hours, preferably, of about 6 hours.

Typically, the heated reaction mixture can be further maintained until the consumption of hydrogen gas is stopped, as determined by an H₂ flowmeter or by decreased pressure in the reaction vessel. This is to ensure complete conversion of the compound of N-methyl-2-(3,4-dimethoxyphenyl)-2-hydroxy-ethylamine and a salt thereof of formula 5 to the compound of N-Methyl-3,4-dimethoxyphenylethylamine and salts thereof of formula 6.

To further convert the compound of N-methyl-2-(3,4-dimethoxyphenyl)-2-hydroxy-ethylamine and a salt thereof of formula 5 to the compound of N-Methyl-3,4-dimethoxyphenylethylamine and salts thereof of formula 6, the reaction mixture is heated to a temperature of about 80° C., preferably of about 85° C. to about 100° C.

Usually, the compound of N-Methyl-3,4-dimethoxyphenylethylamine and salts thereof of formula 6 are obtained during heating for a period of about 20 hours, preferably, of about 20 to about 40 hours, more preferably, of about 30 to about 36 hours.

Preferably, the hydrogenation reaction mixture comprises a salt of N-methyl-2-(3,4-dimethoxyphenyl)-2-hydroxy-ethylamine and a salt thereof of formula 5 or a salt of N-Methyl-3,4-dimethoxyphenylethylamine of formula 6, which can then be recovered. The recovery can be done, for example by filtering the reaction mixture to remove the catalyst, converting the compound of formula 5 or 6 to its free base form, and transforming it back to its salt form, which precipitates.

Preferably, the conversion of the compound of formula 5 or 6 to its free base form is done by using an inorganic base. Preferably, the inorganic base is an alkaline hydroxide, such as sodium hydroxide or potassium hydroxide. More preferably, the alkaline hydroxide is sodium hydroxide.

Typically, the addition of the base provides a basic pH, preferably, of about 12; where in such pH the free base of the compound of formula 5 or 6 is present. To dissolve the obtained free base, this conversion is done in the presence of a water-immiscible organic solvent, such as toluene. The obtained organic phase comprising of the free base of compound of formula 5 or 6 can then be washed with water, concentrated to obtain a residue, which is then dissolved in a solvent selected from the group consisting of acetone, methyl ethyl ketone and methyl isobutyl ketone.

The obtained residue comprises the free base form of formula 5 or 6, can be further acidified if required. Preferably, acidification can be achieved by reacting with a proton donor.

Preferably, the reaction comprises combining a proton donor, such as HCl, HBr or NH₄Cl with the solution in acetone, transforming the free base back to its salt form, i.e., the compound of formula 6, which precipitates and is filtered. Preferably, the proton donor is NH₄Cl.

When NH₄Cl is used a selective precipitation of the compound of formula 6 occurs, i.e., impurity such as N-methyl-4-methoxyphenylethylamine hydrochloride remains in solution in its salt form. Hence, the obtained compound of formula 6 contains less than about 0.02% of N-methyl-4-methoxyphenylethylamine hydrochloride impurity, having the following formula:

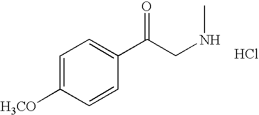

In addition, the obtained HCl salt of N-Methyl-3,4-dimethoxy phenylethylamine of formula 6 is crystalline.

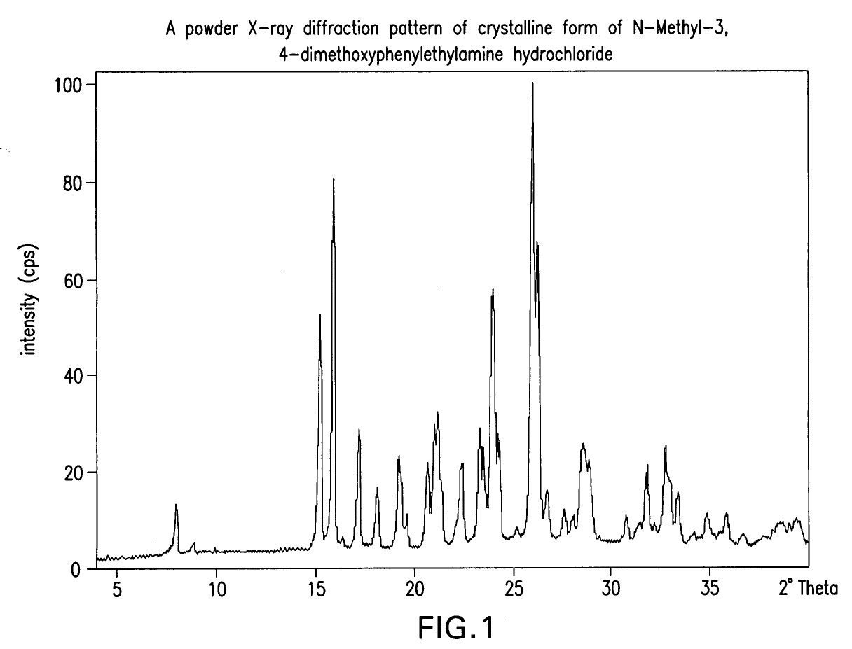

The crystalline form of HCl salt of N-Methyl-3,4-dimethoxy phenylethylamine of formula 6 is characterized by data selected from the group consisting of PXRD pattern having peaks at about 8.0, 15.3, 16.0, 24.0 and 26.0±0.2 degrees 2-theta, and a PXRD pattern as depicted in FIG. 1.

The crystalline form of HCl salt of N-Methyl-3,4-dimethoxyphenylethylamine of formula 6 can be further characterized by PXRD pattern having peaks at about 8.9, 16.4, 17.2, 18.2, 19.2, 19.4, 19.7, 20.7, 21.0, 21.2, 21.4, 22.5, 23.3, 23.6, 24.3, 25.2, 26.3, 26.8, 27.6, 28.1, 28.4, 28.6, 28.9, 30.8, 31.4, 31.8, 32.2, 32.7, 33.0, 33.3, 34.2, 34.9, 35.9, 36.7, 37.7, 38.7, 39.0, 39.3 and 39.6±0.2 degrees 2-theta.

The hydrogenation reaction can also be done with the compound of N-methyl-2-(3,4-dimethoxyphenyl)-2-hydroxy-ethylamine and a salt thereof of formula 5 as the starting material, where the reaction conditions should be modified accordingly.

The obtained compound of formula 6 can then be converted to verapamil of the following formula,

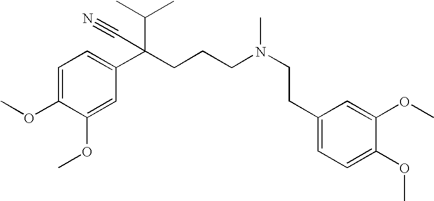

as described in U.S. Pat. No. 4,115,432 and in U.S. Pat. No. 4,350,636.

### EXAMPLES

### PXRD

XRD diffraction was performed on X-Ray powder diffractometer: Philips X'pert Pro powder diffractometer, CuKα radiation, λ=1.5418 Å. X'Celerator detector active length (2 theta)=2.122 mm, laboratory temperature 22-25° C. Zero background sample-holders. Prior to analysis the samples were gently ground by means of mortar and pestle in order to obtain a fine powder. The ground sample was adjusted into a cavity of the sample holder and the surface of the sample was smoothed by means of a cover glass.

### Example 1

### Preparation of N-methyl-3,4-dimethoxyphenylethylamine Hydrochloride (Compound 6 where n is 1 and HX is HCl)

10 g of N-methyl-2-(3,4-dimethoxyphenyl)-2-hydroxy-ethylamine hydrochloride were dissolved in 100 ml of water, then 10.8 g of aluminium tri-chloride were added. The mixture was heated up to 85° C. then 1 g of Pd/C 5% 50% wet was added to the mixture. The suspension so obtained was then hydrogenated under pressure (50 psi) under vigorous agitation for 36 hrs at a temperature of 85° C.

The catalyst was then recovered by filtration maintaining the temperature above 60° C. Then 50 ml of toluene were added and the mixture treated with NaOH 30% (40 ml) till pH12 and clear solution was obtained. The clear aqueous phase, containing sodium aluminium hydroxides was discharged; the toluene solution was washed two times with small quantity of cold water (3 ml) for the sake of removing the un-reacted starting material. The toluene solution was then concentrated under vacuum till residue, the residue was taken up with acetone 40 ml. Hydrochloric acid was bubbled maintaining the mixture temperature below 15° C. to get precipitation of N-methyl-3,4-dimethoxyphenylethylamine hydrochloride in highly pure form, which was cooled to 5° C. and then collected obtaining 9.2 g of N-methyl-3,4-dimethoxyphenylethylamine hydrochloride yield: 98.2%-purity 99.9%.

### Example 2

### Preparation of N-methyl-2-(3,4-dimethoxyphenyl)-2-oxy-ethylamine Hydrochloride (Compound 4 where n is 1 and HY is HCl)

In a vessel load: 120 g of nitrobenzene and 53.3 g of aluminium chloride making attention to the exothermic effect, then 21.2 g of methylaminoacetonitrile hydrochloride were added. Then 27.6 g of veratrole were poured into the mixture. In a few minutes a homogeneous solution was obtained, then hydrochloric acid was bubbled through the solution vigorously for 6 hrs, at a temperature of 25° C.

The mixture was then poured into 320 ml of water under cooling. Then the organic layer was separated at 75° C. and the aqueous layer undergoes to vacuum distillation (50 ml are distilled off) in order to remove the latest traces of solvent. The obtained aqueous layer was then treated with 4.9 g of decolorizing charcoal for 1 hr at 80° C. Then the charcoal was filtered off and the panel washed with 50 ml of water which were joined together with the mother liqueur.

### Example 3

### Preparation of N-methyl-3,4-dimethoxyphenylethylamine Hydrochloride (Compound 6 where n is 1 and HX is HCl)

The solution obtained in example 2), containing 45 g of N-methyl-2-(3,4-dimethoxyphenyl)-2-oxy-ethylamine hydrochloride together with 53.3 g of aluminium tri-chloride and 10.7 g of ammonium chloride, was heated up to 65° C. under hydrogen together with 1 g of Pd/C 5% 50% wet. The suspension so obtained was then hydrogenated under pressure (50 psi) under vigorous agitation for 6 hrs then the temperature was risen up to 85° C. and the hydrogenation was prolonged for 36 hrs, at a temperature of 85° C.

During the reaction time further 4 g of Pd/C 5% 50% wet were added to the mixture.

The catalyst was then recovered by filtration maintaining the temperature above 60° C. Then 250 ml of toluene were added and the mixture treated with NaOH 30% (200 ml) till pH12 and clear solution was obtained. The clear aqueous phase, containing sodium aluminium hydroxides was discharged; the toluene solution was washed twice with small quantity of cold water (15 ml) for the sake of removing the un-reacted starting material. The toluene solution was then concentrated under vacuum till residue, the residue was taken up with acetone 200 ml. Hydrochloric acid was bubbled maintaining the mixture temperature below 15° C. to get precipitation of N-methyl-3,4-dimethoxyphenylethylamine hydrochloride in highly pure form, which was cooled to 5° C. and then collected obtaining 41.8 g of N-methyl-3,4-dimethoxyphenylethylamine hydrochloride yield (two steps): 90.2% purity 99.4%.

### Example 4

### Preparation of Verapamil from N-Methyl-3,4-dimethoxyphenylethylamine and Salt Thereof of Formula 6 According to U.S. Pat. No. 4,115,432 Example 1

(a) 573 g (2.62 mol) of α-isopropyl veratryl cyanide and 481 g (1.1×2.62 mol) of β-chloropropionaldehyde diethyl acetal were dissolved in 2.7 l of dry toluene with heating. 393 g of a 30 percent sodium amide suspension in toluene (117.6 g or 1.15×2.62 mol of NaNH₂) were added drop wise to the boiling solution with vigorous stirring during the course of an hour. After 3 hours the reaction solution was cooled and washed once with 3 l of an ice-water mixture and subsequently washed twice with 1 l of water. The toluene phase was dried and concentrated. Distillation of the residue gives 772 g of α-isopropyl-α-(γ-diethoxypropyl)-veratryl cyanide.

(b) 445 g (1.274 mol) of the substance obtained according to (a) were dissolved in 2.9 l of acetone. Over 45 minutes, 127.5 g (1.1×1.274 mol) of oxalic acid in 1150 ml of water were added drop wise thereto with stirring. The solution was left to stand for 3 hours at 40° C., subsequently cooled to 5° C.-10° C., and adjusted to a pH of 6.0 with a saturated aqueous potassium carbonate solution. The precipitated potassium oxalate was filtered off and the acetone was removed from the filtrate in a rotary evaporator. The oily aldehyde which separates thereby was taken up in diethyl ether and dried over potassium carbonate. After evaporation, 413 g of a yellowish oil was obtained which contains 85 percent of α-isopropyl-α-(γ-oxopropyl)-veratryl cyanide (as the semicarbazone).

(c) 91.1 g of the yellowish oil obtained according to (b) [corresponding to 77.4 g (0.281 mol) of pure α-isopropyl-α-(γ-oxopropyl)-veratryl cyanide] and 54.9 g (0.281 mol) of N-Methyl-3,4-dimethoxyphenylethylamine hydrochloride (the compound of formula 6 where n is 1 and HX is HCl) were dissolved in 400 ml of ethanol and hydrogenated with 10 grams of 10 percent palladium-charcoal at 40° C.-45° C. After conclusion of hydrogen uptake, the catalyst was separated by vacuum filtration and the filtrate was reduced to dryness. The oily residue was dissolved in 500 ml of toluene and washed twice with 250 ml portions of water. The aqueous extracts were discarded. The toluene solution was extracted twice with 150 ml portions of 2 N hydrochloric acid and twice with 150 ml portions of warm water. The aqueous extracts were combined, made alkaline with sodium hydroxide, and extracted with toluene. The toluene extracts were dried, evaporated, and the residue was dissolved in isopropanol. After the introduction of hydrogen chloride gas, 127.0 g of 2-(3,4-dimethoxyphenyl)-5-[2-(3,4-dimethoxyphenyl)ethyl-methyl-amino]-2-(1-methylethyl) pentanenitrile (Verapamil) hydrochloride separates.

### Example 5

### Preparation of N-methyl-2-(3,4-dimethoxyphenyl)-2-hydroxy-ethylamine without Using Aluminium Chloride

10 g of N-methyl-2-(3,4-dimethoxyphenyl)-2-oxy-ethylamine hydrochloride were dissolved in 100 ml of water. The mixture was heated up to 75° C. then 1 g of Pd/C 5% 50% wet was added to the mixture. The suspension so obtained was then hydrogenated under vigorous agitation (50 psi), at a temperature of 75° C. A sample withdrawn and analysed by HPLC after 16 hr shows 99.4% of the compound (5); <0.1% compound (4) and 0.4% of compound (6). After 24 hr the analytical profile shows (5)/(6)=99.2/0.6. After 40 hr the same ratio is 98.8/0.9.

The catalyst was then recovered by filtration maintaining the temperature above 60° C. Then 50 ml of toluene were added and the mixture was treated with NaOH 30% (40 ml) till pH12 and clear solution was obtained. The clear aqueous phase, containing sodium aluminium hydroxides was discharged; the toluene solution was then concentrated under vacuum till residue.

8.2 g of N-methyl-2-(3,4-dimethoxyphenyl)-2-hydroxy-ethylamine were recovered. Yield: 95.1%-purity 99.0% (compound 6 content=0.4%)

### Example 6

### Preparation of N-methyl-3,4-dimethoxyphenylethylamine Hydrochloride (Compound 6 where n is 1 and HX is HCl) with Postponed Addition of Aluminium Chloride

### Extension of Example 5

10 g of N-methyl-2-(3,4-dimethoxyphenyl)-2-oxy-ethylamine hydrochloride were dissolved in 100 ml of water. The mixture was heated up to 75° C. then 1 g of Pd/C 5% 50% wet is added to the mixture. The suspension so obtained was then hydrogenated under vigorous agitation. A sample withdrawn after 16 hr shows 99.4% of the compound (5); <0.1% compound (4) and 0.4% of compound (6). After 24 hr the analytical profile shows (5) (6) 99.2/0.6. After 40 hr the same ratio is 98.8/0.9. Then 10.8 g of aluminium tri-chloride are added. After further 20 hr the analytical profile shows (5)/(6) 89.7/10.0. After globally 90 hr the analytical profile shows (5)/(6) 58.1/40.8. Then pressure of 50 psi of Hydrogen was applied and the reaction was over after 10-15 hr.

The catalyst was then recovered by filtration maintaining the temperature above 60° C. Then 50 ml of toluene were added and the mixture treated with NaOH 30% (40 ml) till pH12 and clear solution was obtained. The clear aqueous phase, containing sodium aluminium hydroxides was discharged; the toluene solution was washed two times with small quantity of cold water (3 ml) for the sake of removing the un-reacted starting material. The toluene solution was then concentrated under vacuum till residue, the residue was taken up with acetone 40 ml. Hydrochloric acid was bubbled maintaining the mixture temperature below 15° C. to get precipitation of N-methyl-3,4-dimethoxyphenylethylamine hydrochloride in highly pure form, which was cooled to 5° C. and then collected obtaining 9.2 g of N-methyl-3,4-dimethoxyphenylethylamine hydrochloride yield: 97.9%-purity 99.8%.

### Example 7

### Preparation of N-methyl-3,4-dimethoxyphenylethylamine Hydrochloride (Compound 6 where n is 1 and HX is HCl) Using Broensted Acid (Hydrochloric Acid) Instead of Lewis Acid (Aluminium Chloride)

### Extension of Example 5

10 g (0.04 mol) of N-methyl-2-(3,4-dimethoxyphenyl)-2-oxy-ethylamine hydrochloride were dissolved in 100 ml of water. The mixture was heated up to 75° C. then 1 g of Pd/C 5% 50% wet was added to the mixture. The suspension so obtained was then hydrogenated under vigorous agitation. A sample withdrawn and analysed by HPLC after 24 hr shows 99.4% of the compound (5); <0.1% compound (4) and 0.5% of compound (6). Then 3.0 g (0.08 mol) of HCl were added and the solution hydrogenated for further 19 hr. A sample withdrawn and analysed by HPLC shows ratio (5)/(6) of 76.9/21.3 and 1.8% of by-products (0.7% dimer precursor and 0.3% dimer). Then further HCl 3.0 g (0.08 mol) were added and a sample was analysed after 24 Hr showing: ratio (5)/(6) of 45.1/47.0 and 7.8% of by-products (1.0% dimer precursor and 4.1% dimer). A further addition of HCl (6 g 0.16 mol) was not able to complete the reaction and after further 72 Hr the analytical profile is: ratio (5)/(6) of 5.8/55.9 and 38.3% of by-products (0.6% dimer precursor and 25.4% dimer).

### Example 8

### Preparation of N-methyl-2-(3,4-dimethoxyphenyl)-2-oxy-ethylamine Hydrochloride (Compound 4 where n is 1 and HY is HCl) Polluting the Starting Material “veratrole” with 2% w/w of Anisole

In a vessel load: 120 g of nitrobenzene and 53.3 g of aluminium chloride making attention to the exothermic effect, then 21.2 g of methylaminoacetonitrile hydrochloride were added. Then 27.1 g of veratrole and 0.6 g of anisole were poured into the mixture. In a few minutes a homogeneous solution was obtained, then hydrochloric acid was bubbled through the solution vigorously for 6 hrs at a temperature of 25° C.

The mixture was then poured into 320 ml of water under cooling. Then the organic layer was separated at 75° C. and the aqueous layer underwent to vacuum distillation (50 ml are distilled off) in order to remove the latest traces of solvent. The obtained aqueous layer was then treated with 4.9 g of decolorizing charcoal for 1 hr at 80° C. Then the charcoal was filtered off and the panel washed with 50 ml of water which were joined together with the mother liqueur.

### Example 9

### Preparation of N-methyl-3,4-dimethoxyphenylethylamine Hydrochloride (Compound 6 where n is 1 and HX is HCl)

The solution obtained in example 8), containing 45 g of N-methyl-2-(3,4-dimethoxyphenyl)-2-oxy-ethylamine hydrochloride (plus 0.9 g of N-methyl-2-(4-methoxyphenyl)-2-oxy-ethylamine hydrochloride) together with 53.3 g of aluminium tri-chloride and 10.7 g of ammonium chloride, was heated up to 65° C. under hydrogen together with 1 g of Pd/C 5% 50% wet. The suspension so obtained was then hydrogenated under pressure (50 psi) under vigorous agitation for 6 hrs then the temperature was risen up to 85° C. and the hydrogenation was prolonged for 36 hrs.

During the reaction time further 4 g of Pd/C 5% 50% wet were added to the mixture.

The catalyst was then recovered by filtration maintaining the temperature above 60° C. Then 250 ml of toluene were added and the mixture treated with NaOH 30% (200 ml) till pH12 and clear solution was obtained. The clear aqueous phase, containing sodium aluminium hydroxides was discharged; the toluene solution was washed twice with small quantity of cold water (15 ml) for the sake of removing the un-reacted starting material. The toluene solution was then concentrated under vacuum till residue, the residue was taken up with acetone 200 ml. Hydrochloric acid was bubbled maintaining the mixture temperature below 15° C. to get precipitation of N-methyl-3,4-dimethoxyphenylethylamine hydrochloride, which was cooled to 5° C. and then collected obtaining 41.8 g of N-methyl-3,4-dimethoxyphenylethylamine hydrochloride yield (two steps): 90.2% purity 97.9% N-methyl-4-methoxyphenylethylamine hydrochloride impurity content: 1.5% w/w.

### Example 10

### Preparation of N-methyl-3,4-dimethoxyphenylethylamine Hydrochloride (Compound 6 where n is 1 and HX is HCl)

### Alternative Isolation

The solution obtained in example 8), containing 45 g of N-methyl-2-(3,4-dimethoxyphenyl)-2-oxy-ethylamine hydrochloride (plus 0.9 g of N-methyl-2-(4-methoxyphenyl)-2-oxy-ethylamine hydrochloride) together with 53.3 g of aluminium tri-chloride and 10.7 g of ammonium chloride, was heated up to 65° C. under hydrogen together with 1 g of Pd/C 5% 50% wet. The suspension so obtained was then hydrogenated under pressure (50 psi) under vigorous agitation for 6 hrs then the temperature was risen up to 85° C. and the hydrogenation was prolonged for 36 hrs.

During the reaction time further 4 g of Pd/C 5% 50% wet were added to the mixture.

The catalyst was then recovered by filtration maintaining the temperature above 60° C. Then 250 ml of toluene were added and the mixture treated with NaOH 30% (200 ml) till pH12 and clear solution was obtained. The clear aqueous phase, containing sodium aluminium hydroxides was discharged; the toluene solution was washed twice with small quantity of cold water (15 ml) for the sake of removing the un-reacted starting material. The toluene solution was then concentrated under vacuum till residue, the residue was taken up with acetone 90 ml. Then ammonium chloride 9.6 g were added under agitation and the suspension was maintained in agitation for 4-6 hrs. Then 40 ml of acetone were added to the mixture and the solid was collected by filtration and it was then washed with 40 ml of acetone, obtaining 38.1 g of dry N-methyl-3,4-dimethoxyphenylethylamine hydrochloride yield (two steps): 82.2% purity 99.9%. N-methyl-4-methoxyphenylethylamine hydrochloride impurity content: 0.02% w/w.

### Example 11

### Preparation of N-methyl-3,4-dimethoxyphenylethylamine Hydrochloride (Compound 6 where n is 1 and HX is HCl)

The solution obtained in example 2), containing 45 g of N-methyl-2-(3,4-dimethoxyphenyl)-2-oxy-ethylamine hydrochloride together with 53.3 g of aluminium tri-chloride and 10.7 g of ammonium chloride, was heated up to 62° C. under hydrogen together with 0.5 g of Pd/C 10% 50% wet. The suspension so obtained was then hydrogenated under pressure (50 psi) under vigorous agitation for 6 hrs while the temperature was smoothly risen up to 70° C. and the hydrogenation was prolonged till no further consumption of hydrogen was observed (36 hrs). During the reaction time further 4 g of a composite catalyst Pd—Ru/C 4.5%-0.5%, 50% wet were added to the mixture.

The catalyst was then recovered by filtration maintaining the temperature above 60° C. Then 250 ml of toluene were added and the mixture treated with NaOH 30% (200 ml) till pH12 and clear solution was obtained. The clear aqueous phase, containing sodium aluminium hydroxides was discharged; the toluene solution was washed twice with small quantity of cold water (15 ml) for the sake of removing the un-reacted starting material.

The toluene solution was then concentrated under vacuum till residue, the residue was taken up with acetone 90 ml. Then ammonium chloride 9.6 g were added under agitation and the suspension was maintained in agitation for 4-6 hrs. Then 40 ml of acetone were added to the mixture and the solid was collected by filtration and it was then washed with 40 ml of acetone, obtaining 42.3 g of dry N-methyl-3,4-dimethoxyphenylethylamine hydrochloride yield (two steps): 92.0% purity 99.99%. N-methyl-4-methoxyphenylethylamine hydrochloride impurity content: 0.01% w/w.

### Example 12

The preparation was performed following the recipe shown in example 11 being the only difference the catalyst used that was Ruthenium 5% on charcoal. After 50 hrs of hydrogenation, only 5% of conversion to compound (5) and 6% of compound (6) was found after HPLC check.

1. A process for preparing N-Methyl-3,4-dimethoxyphenylethylamine and a salt thereof of formula 6. 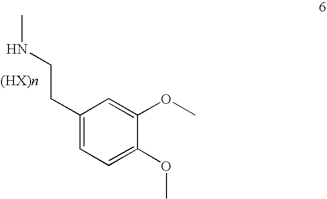
comprising reacting N-methyl-2-(3,4-dimethoxyphenyl)-2-oxy-ethylamine and a salt thereof of formula 4 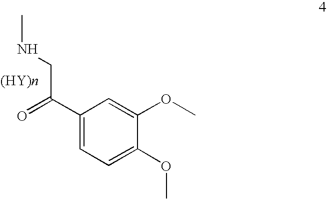
or N-methyl-2-(3,4-dimethoxyphenyl)-2-hydroxy-ethylamine and a salt thereof of formula 5 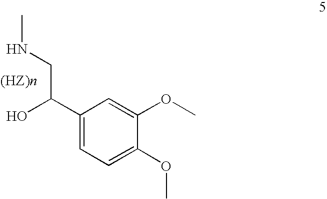
with hydrogen gas, a palladium hydrogenation catalyst and a Lewis acid; wherein n is either 0 or 1, when n of HY or HZ is 0 the reaction comprises also HCl or HBr; and HX, HY and HZ are independently an acid selected from a group consisting of: HCl and HBr, and combination thereof.

2. The process of claim 1, wherein an aqueous mixture comprising the compound of N-methyl-2-(3,4-dimethoxyphenyl)-2-oxy-ethylamine and a salt thereof of formula 4 or N-methyl-2-(3,4-dimethoxyphenyl)-2-hydroxy-ethylamine and a salt thereof of formula 5 is reacted with Lewis acid.

3. The process of claim 2, wherein the concentration of the Lewis acid in said aqueous mixture is of about 5% to about 15% by weight per water.

4. The process of claim 1, wherein the Lewis acid is a metallic Lewis acid.

5. The process of claims 1, wherein the Lewis acid is a metallic Lewis acid containing a halogen counter ion.

6. The process of claim 5, wherein the halogen counter ion is Cl⁻ or Br⁻.

7. The process of claim 5, wherein the metallic Lewis acid containing a halogen counter ion is selected from the group consisting of: Aluminium, Titanium, Iron and Zinc Lewis acids.

8. The process of claim 7, wherein the metallic Lewis acid containing a halogen counter ion is selected from the group consisting of: AlCl3, AlBr3, FeCl3, FeBr3, TiCl4, ZnCl2 and ZnBr2.

9. The process of claim 8, wherein the metallic Lewis acid containing a halogen counter ion is AlCl3.

10. The process of claim 1, wherein about 1 to about 3 mole equivalent of Lewis acid per mole equivalent of the compound of N-Methyl-2-(3,4-dimethoxyphenyl)-2-oxy-ethylamine of formula 4 are reacted.

11. The process of claim 1, wherein a mixture comprising the starting compound of formula 4, the Lewis acid, and optionally, an acid and/or acidic salt, is heated to a temperature of about 55° C. to about 100° C., providing a suspension.

12. The process of claim 1, wherein the palladium hydrogenation catalyst is selected from the group consisting of: Pd (OH)2, PdCl2, Pd/C Pd/graphite, Palladium on activated Charcoal and palladium catalysts that is polluted with about 5% (w/w) of Ruthenium.

13. The process of claim 1, wherein the palladium hydrogenation catalyst is Palladium on activated Charcoal or Pd/C.

14. The process of claim 1, wherein the total amount of the palladium hydrogenation catalyst is added at about 0.1% to about 10% by weight per weight of the starting compound of N-Methyl-2-(3,4-dimethoxyphenyl)-2-oxy-ethylamine of formula 4.

15. The process of claim 14, wherein the palladium hydrogenation catalyst is wet.

16. The process of claim 1, wherein the hydrogenation reaction is done upon heating to a temperature of less than about 80° C.

17. The process of claim 1, wherein the hydrogenation reaction is done upon heating to a temperature of about 80° C. to about 100° C.

18. The process of claim 16, wherein the salt of N-Methyl-2-(3,4-dimethoxyphenyl)-2-hydroxy-ethylamine of formula 5, is obtained.

19. The process of claim 17, wherein the salt of N-Methyl 3,4-dimethoxyphenylethylamine of formula 6 is obtained.

20. The process of claim 18, wherein the salt of N-Methyl-2-(3,4-dimethoxyphenyl)-2-hydroxy-ethylamine of formula 5 is recovered.

21. The process of claim 19, wherein the salt of N-Methyl 3,4-dimethoxyphenylethylamine of formula 6 is converted to the free base.

22. The process of claim 21, wherein the free base N-Methyl 3,4-dimethoxyphenylethylamine of formula 6 is dissolved in a solvent selected from the group consisting of acetone, methyl ethyl ketone and methyl isobutyl ketone.

23. The process of claim 22, wherein the solution is combined with a proton donor transforming the free base back to its salt form.

24. The process of claim 23, wherein the proton donor is selected from the group consisting of HCl, HBr or NH4Cl.

25. The process of claim 23, wherein the salt of N-Methyl 3,4-dimethoxyphenylethylamine of formula 6 is recovered.

26. A process to prepare verapamil of the following formula: 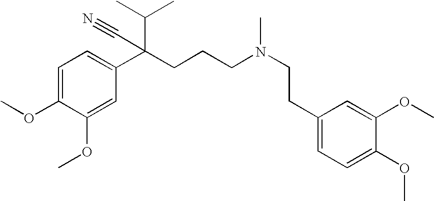
comprising preparing the compound of N-Methyl 3,4-dimethoxyphenylethylamine of formula 6 according to the process of claim 1, and converting it to verapamil.
